(19) United States
(12) Patent Application Publication
(10) Pub. No.: US 2007/0249738 A1
(43) Pub. Date: Oct. 25, 2007

(54) PREMIXED PARTIAL OXIDATION SYNGAS GENERATOR

(76) Inventors: Joel Meier Haynes, Niskayuna, NY (US); John Thomas Herbon, Rexford, NY (US); Anthony John Dean, Scotia, NY (US); Mohamed Ahmed Ali, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/380,059

(22) Filed: Apr. 25, 2006

(51) Int. Cl. C07C 27/06; B01J 19/00; B01J 3/00

(52) U.S. Cl. 518/702; 422/165

(57) ABSTRACT

A premixed partial oxidation (POₓ) syngas generator is provided. The syngas generator includes a premixing device configured to mix a fuel stream and oxygen in a premixing region to form a gaseous pre-mix. The premixing device includes a fuel inlet configured to introduce the fuel stream within the premixing device and a flow conditioning device configured to pre-condition the fuel stream. The premixing device also includes an oxygen inlet configured to introduce oxygen into the fuel stream to facilitate premixing of the fuel stream and oxygen in the premixing region located downstream of the flow conditioning device. The syngas generator also includes a combustion chamber configured to combust the gaseous pre-mix from the premixing device to produce a synthesis gas enriched with carbon monoxide and hydrogen gas.

### BACKGROUND

The invention relates generally to syngas generators, and more particularly to a syngas generator based on premixed partial oxidation combustion.

Currently industrial plants are built around the globe to produce synthesis gas for use in a variety of applications including conversion of natural gas to useful liquid fuels, generation of hydrogen-enriched gases and other processes. Typically, synthesis gases produced in a gas to liquid plant are supplied to a Fischer Tropsch processing unit for catalytically converting the quenched synthesis gas into a long-chain hydrocarbon fluid. Further, the long-chain hydrocarbon fluid mixture is fractionated into at least one useful product through an upgrading process.

In certain traditional systems, synthesis gases are produced through diffusion combustion of reactants in a syngas generator. Unfortunately, the diffusion combustion requires a substantially long residence time to ensure that the products of the diffusion flame achieve near equilibrium products at the exit of a syngas generator. Moreover, the resulting products are required to be cleaned to remove carbon deposits in the products followed by cooling of the cleaned products for further processing.

Certain other systems employ autothermal reforming or catalytic partial oxidation techniques for generating the synthesis gases. However, such techniques require catalysts that have substantially high capital and operating costs.

Accordingly, there is a need for a syngas generator that has a high conversion efficiency of natural gas to syngas products. Furthermore, it would be desirable to provide a syngas generator with reduced complexity and size. Lowering the overall complexity of these systems will drastically reduce the capital and operating costs for synthesis gas generation.

### BRIEF DESCRIPTION

Briefly, according to one embodiment, a premixed partial oxidation (POₓ) syngas generator is provided. The syngas generator includes a premixing device configured to mix a fuel stream and oxygen in a premixing region to form a gaseous pre-mix. The premixing device includes a fuel inlet configured to introduce the fuel stream within the premixing device and a flow conditioning device configured to pre-condition the fuel stream. The premixing device also includes an oxygen inlet configured to introduce oxygen into the fuel stream to facilitate premixing of the fuel stream and oxygen in the premixing region located downstream of the flow conditioning device. The syngas generator also includes a combustion chamber configured to combust the gaseous pre-mix from the premixing device to produce a synthesis gas enriched with carbon monoxide and hydrogen gas.

In another embodiment, a gas to liquid system is provided. The gas to liquid system includes an air separation unit configured to separate oxygen from air and a gas processing unit configured to prepare a fuel stream for combustion. The gas to liquid system also includes a syngas generator for reacting oxygen with the fuel stream at an elevated temperature and pressure to produce a synthesis gas enriched with carbon monoxide and hydrogen gas and a premixing device disposed upstream of the syngas generator and configured to mix the fuel stream and oxygen. The premixing device includes a fuel inlet configured to introduce the fuel stream within the premixing device and a flow conditioning device configured to pre-condition the fuel stream. The premixing device also includes an oxygen inlet configured to introduce oxygen into the fuel stream to facilitate premixing of fuel stream and oxygen in a premixing region located downstream of the flow conditioning device.

In another embodiment, a method of generating a synthesis gas is provided. The method includes introducing a fuel stream within a premixing device and preconditioning the fuel stream through a flow conditioning device. The method also includes introducing an oxygen stream downstream of the flow conditioning device to facilitate premixing of the fuel stream and oxygen to form a gaseous pre-mix and forming the synthesis gas through partial oxidation of the gaseous pre-mix.

### DRAWINGS

These and other features, aspects, and advantages of the present invention will become better understood when the following detailed description is read with reference to the accompanying drawings in which like characters represent like parts throughout the drawings, wherein:

FIG. 1 is a diagrammatical illustration of a gas to liquid system having a syngas generator with a premixing device in accordance with aspects of the present technique;

FIG. 2 is a diagrammatical illustration of an exemplary configuration of a premixed partial oxidation (POₓ) syngas generator employed in the gas to liquid system of FIG. 1 in accordance with aspects of the present technique;

FIG. 3 is a diagrammatical illustration of another exemplary configuration of a premixed partial oxidation (POₓ) syngas generator employed in the gas to liquid system of FIG. 1 in accordance with aspects of the present technique;

FIG. 4 is a cross-sectional view of a premixed POₓ syngas generator employed in the gas to liquid system of FIG. 1 in accordance with aspects of the present technique;

FIG. 5 is a cross-sectional view of another exemplary configuration of the premixed POₓ syngas generator or combustor employed in the gas to liquid system of FIG. 1.

FIG. 6 is a diagrammatical illustration of a premixing device employed in the syngas generator of FIGS. 4 and 5 in accordance with aspects of the present technique;

FIG. 7 is a diagrammatical illustration of an exemplary configuration of a premixing device having a flow conditioning device in accordance with aspects of the present technique;

FIG. 8 is a diagrammatical illustration of exemplary configurations of the premixing device of FIG. 7 having different oxygen injection locations in accordance with aspects of the present technique;

FIG. 9 is a diagrammatical illustration of another exemplary configuration of a premixing device having a flow conditioning device in accordance with aspects of the present technique;

FIG. 10 is a diagrammatical illustration of an exemplary configuration 180 of the premixing device of FIG. 9 in accordance with aspects of the present technique; and

FIG. 11 represents exemplary results illustrating H2:CO ratio with % carbon monoxide (CO) yield for the syngas generator 12 of FIG. 1 in accordance with aspects of the present technique.

### DETAILED DESCRIPTION

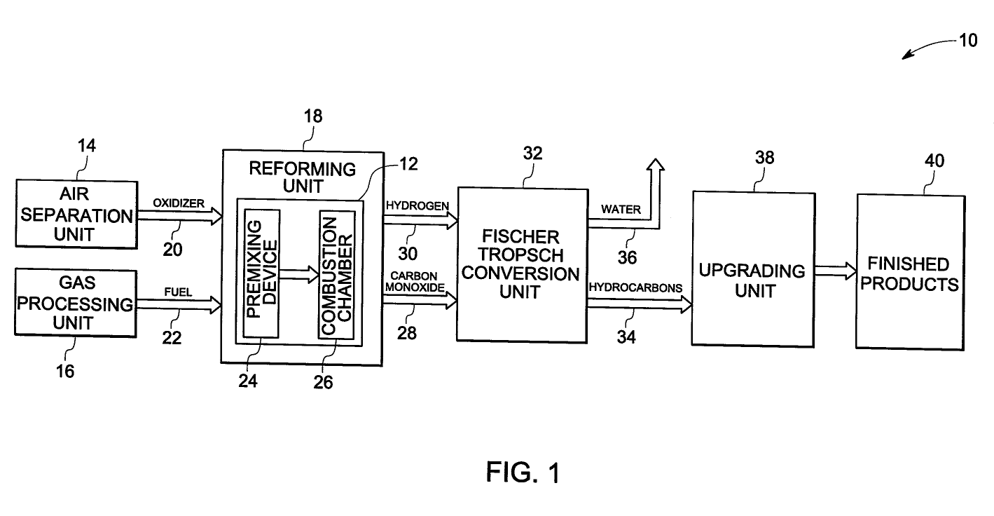

As discussed in detail below, embodiments of the present technique function to enhance conversion efficiency and decrease capital cost of syngas generators and systems. In particular, the present technique employs premixed partial oxidation combustion in a syngas generator that operates with a substantially high fuel to oxygen (or oxidizer) ratio resulting in a syngas composition enriched with carbon monoxide (CO) and hydrogen (H₂). Turning now to the drawings and referring first to FIG. 1 a gas to liquid system 10 having a syngas generator 12 is illustrated. The gas to liquid system 10 typically includes an air separation unit 14 and a gas processing unit 16. The air separation unit 14 separates air into nitrogen (N₂), oxygen (O₂) and other gases. Further, the gas processing unit 16 is configured to prepare a fuel stream for combustion. In particular, the gas processing unit 16 prepares raw natural gas for conversion in a reforming unit 18 by filtering and reducing the levels of impurities such as sulfur.

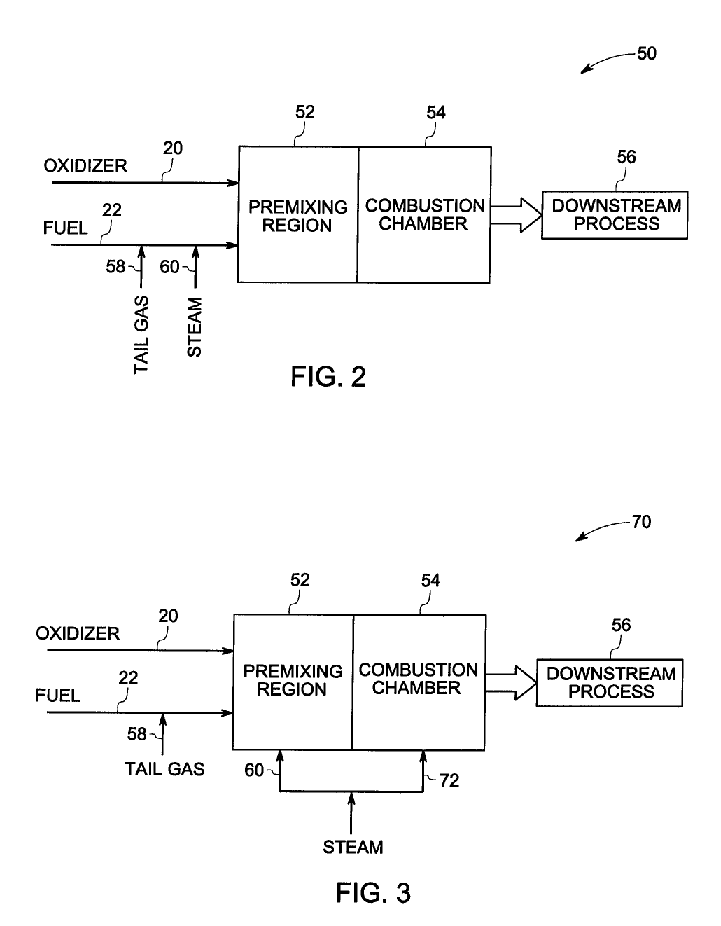

In the illustrated embodiment, the reforming unit 18 includes the syngas generator 12 for reacting an oxidizer such as oxygen 20 and a fuel stream 22 from the air separation and gas processing units 14 and 16, respectively, to produce a synthesis gas. The syngas generator 12 includes a premixing device 24 that is configured to mix the fuel stream 22 and oxygen 20 to form a gaseous pre-mix. Further, the syngas generator 12 includes a combustion chamber 26 configured to combust the gaseous pre-mix from the premixing device 24 to produce synthesis gas enriched with carbon monoxide 28 and hydrogen 30. The gas to liquid system 10 includes a Fischer-Tropsch processing unit 32 for receiving quenched synthesis gas from the reforming unit 18 and for catalytically converting the quenched synthesis gas into hydrocarbons 34 and water 36. In addition, the gas to liquid system 10 includes an upgrading unit 38 for fractionating the hydrocarbons 34 from the Fischer Tropsch conversion unit into at least one useful product 40. Examples of product 40 include synthetic diesel fuel, synthetic kerosene, ethanol, dimethyl ether, naptha and combinations thereof. In accordance with the present techniques, the syngas generator 12 employs premixed partial oxidation combustion that will be described below with reference to FIGS. 2-4.

FIG. 2 is a diagrammatical illustration of an exemplary configuration of a premixed partial oxidation (POₓ) syngas generator 50 employed in the gas to liquid system 10 of FIG. 1. In this exemplary embodiment, the syngas generator 50 includes a premixing region 52 and a combustion chamber 54. In operation, oxidizer 20 and fuel stream 22 are mixed in the premixing region 52 via a premixing device 24 (see FIG. 1) to form a gaseous pre-mix. It should be noted that a plurality of premixing devices 24 may be employed in the syngas generator 50 to deliver the gaseous pre-mix into the combustion chamber 54. As will be appreciated by one skilled in the art the staging of flows to the individual premixing devices 24 may be employed during a start-up condition to enhance stable combustion and also to reduce syngas generator oscillations. In one embodiment, the oxidizer 20 comprises oxygen and the fuel stream 22 comprises natural gas.

The gaseous pre-mix formed in the premixing region 52 is combusted in the combustion chamber 54 at elevated temperature and pressure to form synthesis gas, which in turn, is directed to a downstream process 56 for further processing. In certain embodiments, a stable combustion in the combustion chamber 54 is achieved by a combination of swirling flow and bluff body stabilization. In one embodiment, the syngas generator 50 comprises a rich premixed natural gas combustion system. Alternatively, the syngas generator 50 comprises a rich premixed natural gas combustion system. In one embodiment, the combustion chamber 54 is coated with a catalyst to promote syngas formation.

In certain embodiments, a tail gas 58 may be added to the fuel stream 22 to improve the overall conversion efficiency of the gas to liquid system 10. The tail gas 58 may include a fuel-bearing gas that is recycled from the downstream process 56. For example, in one embodiment in the gas to liquid system 10 (see FIG. 1) the tail gas is a gas phase product from the Fischer Tropsch processing unit 32 (see FIG. 1). Similarly, in certain other embodiments, the fuel stream 22 may be augmented with steam 60 to control the H2:CO ratio of the generated syngas. Further, the steam 60 may also be used to regulate the syngas temperature.

FIG. 3 is a diagrammatical illustration of another exemplary configuration 70 of a premixed partial oxidation (POₓ) syngas generator. As described earlier with reference to FIG. 2, the oxidizer 20 and the fuel stream 22 are mixed in the premixing region 52 to form the gaseous pre-mix that is subsequently combusted in the combustion chamber 54 to produce synthesis gas. In one embodiment, the tail gas 58 is added to the fuel stream 22 for increasing the overall efficiency of the GTL process. In certain embodiments, steam 60 is added to the fuel stream for moderating the temperature within the premixing region 52 and for increasing the H2:CO ratio in the generated syngas. In the illustrated embodiment, steam 72 is also injected into the combustion chamber 54 to increase the H2:CO ratio in the generated syngas.

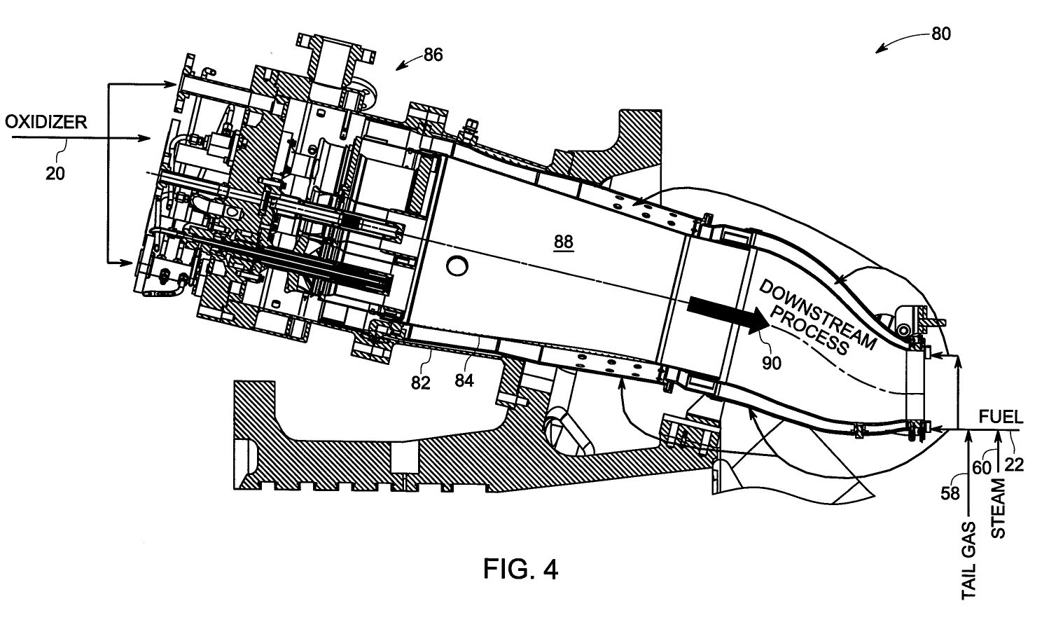

FIG. 4 is an exemplary cross-sectional view of a premixed POₓ syngas generator or combustor 80 employed in the gas to liquid system 10 of FIG. 1. The combustor 80 includes a combustor housing 82 and a combustor liner 84 disposed within the housing 82. In operation, the fuel stream 22 is introduced and premixed with the oxidizer 20 via the premixing device 24 (see FIG. 1) within a premixing region 86 within the housing 82. In this embodiment the fuel stream 22 includes natural gas and the oxidizer 20 includes oxygen. In certain embodiments, the fuel stream includes a hydrocarbon such as methane and may also contain steam, carbon dioxide (CO₂) and hydrogen (H₂). Similarly, the oxidizer 20 may include oxygen with steam or carbon dioxide (CO₂). In the illustrated embodiment, the natural gas 22 is preconditioned through the premixing device 24 and oxygen 20 is introduced in a transverse direction to facilitate the premixing of natural gas 22 and oxygen 20 to form the gaseous pre-mix. It should be noted that the flow conditioning of the fuel stream 22 and subsequent introduction of oxygen 20 enables fast mixing of the fuel stream 22 and oxygen 20. In one embodiment, a premixing residence time of the syngas generator 80 is in the range between about 0.5 ms to about 100 ms. Moreover, a ratio of the number of oxygen atoms to the number of carbon atoms in the fuel stream 22 in the premixing region 86 is typically in the range between about 0.6 to about 1.6.

The gaseous pre-mix formed in the premixing region 86 is combusted in a combustion chamber 88 at elevated temperature and pressure to form synthesis gas that is directed to a downstream process 90 for further processing. In this embodiment, the syngas generator 80 is operated at a pressure of between about 25 atmospheres to about 80 atmospheres of absolute pressure. In certain embodiments, a pilot flame such as a fuel nozzle with a relatively low degree of premixing may be employed to initiate flame during start-up and to ensure stable combustion in the combustion chamber 88. It should be noted that the combustion of substantially premixed reactants in the syngas generator 80 leads to a compact reaction zone that achieves near-equilibrium composition and negligible formation of solid carbon in the reaction zone. As will be appreciated by one skilled in the art the synthesis gas produced by the POₓ syngas generator 80 described above may be utilized by industrial plants that require flows rich in CO and H2. Examples of such applications include gas to liquid plants, hydrogen generation and carbon dioxide sequestration.

In certain embodiments, tail gas 58 may be introduced into the fuel stream 22 to further improve the conversion efficiency of the plant. Similarly steam 60 may be introduced into the fuel stream 26 for increasing the H2:CO ratio and reducing soot formation. Further, the combustion chamber 88 may be cooled with gas flowing on the backside of the syngas generator liner 84. For example, the combustion chamber 88 may be cooled through one of the streams used in the process such as the oxidizer, or the fuel stream in a reverse flow configuration. Alternatively, another process gas such as steam or nitrogen may be utilized for cooling the combustion chamber 88.

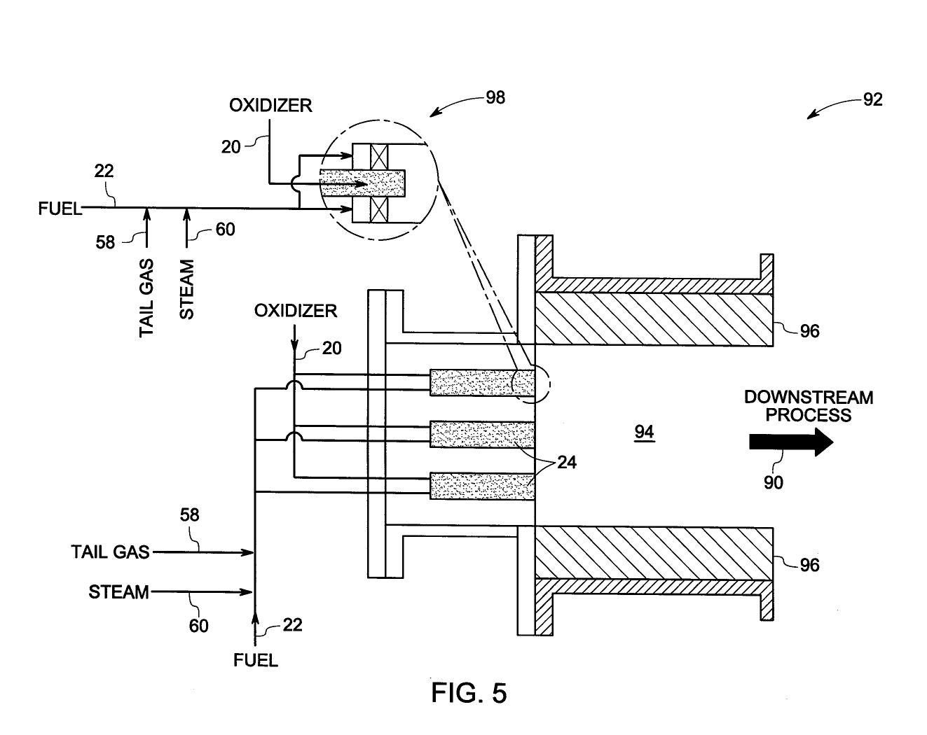

FIG. 5 is a cross-sectional view of another exemplary configuration 92 of the premixed POₓ syngas generator or combustor employed, for example, in the gas to liquid system 10 of FIG. 1. In the illustrated embodiment, the combustor 92 includes a pressure vessel or a reactor 94 disposed within ceramic lined walls 96. Further, the combustor 92 includes a plurality of premixing devices 24 disposed upstream of the reactor 94 for premixing the fuel stream 22 and the oxidizer 20. In this embodiment, the combustor 92 includes three premixing devices 24. However a greater or a lesser number of premixing devices 24 may be envisaged. In this embodiment the fuel stream 22 includes natural gas and the oxidizer 20 includes oxygen. Further, as described above with reference to FIG. 4, the natural gas 22 is preconditioned through each of the premixing devices 24 and oxygen 20 is introduced in a transverse direction to facilitate the premixing of natural gas 22 and oxygen 20 to form the gaseous pre-mix. Subsequently, the gaseous pre-mix is combusted in the reactor 94 at elevated temperature and pressure to form synthesis gas that is directed to the downstream process 90 for further processing.

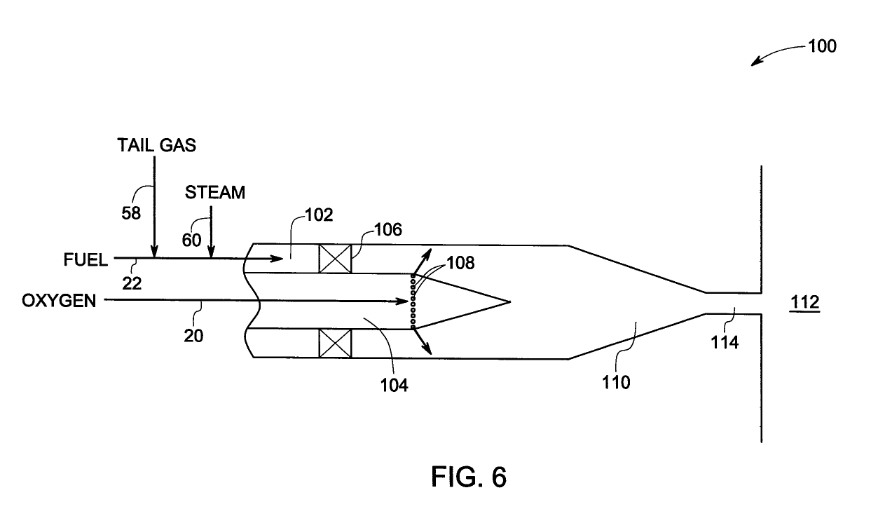

In certain embodiments, tail gas 58 may be introduced into the fuel stream 22 to further improve the conversion efficiency of the plant. Similarly steam 60 may be introduced into the fuel stream 26 for increasing the H2:CO ratio and for reducing soot formation. Further, the operation of the plurality of the premixing devices 24 may be selectively controlled via a controller (not shown) based upon a desired conversion efficiency of the plant. In one embodiment, the premixing device 24 employed for premixing of the fuel stream 22 and oxygen 20 is illustrated in the detailed view 98. In this exemplary embodiment, the premixing device 98 includes fuel inlet to introduce the fuel stream 22 within the premixing device and the fuel stream 22 is pre-conditioned via a plurality of swirlers. Further, the premixing device 98 also includes oxygen inlet to introduce oxygen 20 within the centerbody of the premixing device 98. Exemplary configurations of the premixing device 98 will be explained in a greater detail below with reference to FIGS. 6-10.

FIG. 6 is a diagrammatical illustration of an exemplary configuration 100 of the premixing device employed in the syngas generators 80 and 92 of FIGS. 4 and 5. In the illustrated embodiment, the premixing device 100 includes a fuel inlet 102 configured to introduce the fuel stream 22 within the premixing device 100. In addition, the premixing device 100 includes an oxygen inlet 104 configured to introduce oxygen 20 within the premixing device 100. Further, a flow conditioning device 106 is employed to pre-condition the fuel stream 22 prior to introduction of oxygen within the premixing device 100. In this exemplary embodiment, the flow conditioning device 106 includes a plurality of swirler vanes configured to provide a swirl movement to the fuel stream 22. In an alternate embodiment, the flow conditioning device 106 includes a nozzle configured to accelerate the fuel stream 22 to a desired velocity. However, other types of flow conditioning devices for pre-conditioning the fuel stream 22 are envisaged.

In operation, the fuel stream 22 is pre-conditioned via the plurality of swirler vanes 106. Further, oxygen 20 is introduced in a transverse direction to the direction of injection of the fuel stream 22 via the oxygen inlet 104. In the illustrated embodiment, oxygen 20 is injected at a location 108 disposed downstream of the plurality of swirler vanes 106. In one embodiment, oxygen 20 is introduced through a plurality of holes disposed on each of the plurality of swirler vanes 106. In this embodiment, the pressure drop across the plurality of holes for introducing oxygen 20 is less than 5%. Alternatively, oxygen 20 may be introduced through a center body or walls of the premixing device 100. In one embodiment, oxygen 20 is injected at an angle that has a component perpendicular to the direction of flow. Furthermore, The injection holes may also introduce swirl around the axis of the centerbody of the premixing device 100. The pre-conditioned fuel stream 22 and oxygen 20 are mixed in a premixing region 110 to form a gaseous pre-mix that is further directed to a combustion chamber 112 through an exit 114. In the illustrated embodiment, the premixing region 110 is designed to resist flameholding even in the presence of an ignition source by minimizing recirculation zones.

In this exemplary embodiment, the temperature of the fuel stream 22 is between about 400° F. to about 1300° F. and the temperature of oxygen 20 is between about 200° F. to about 500° F. Further, the ratio of an effective area of the oxygen inlet 104 and an effective area of the flow conditioning device 106 is between about 0.1 to about 0.5. In an exemplary embodiment, the flow conditioning device 106 is configured to introduce the fuel stream 22 in a rich premixed natural gas combustion system and oxygen 20 is introduced within the premixing device 100 in about 1/2 portions by volume.

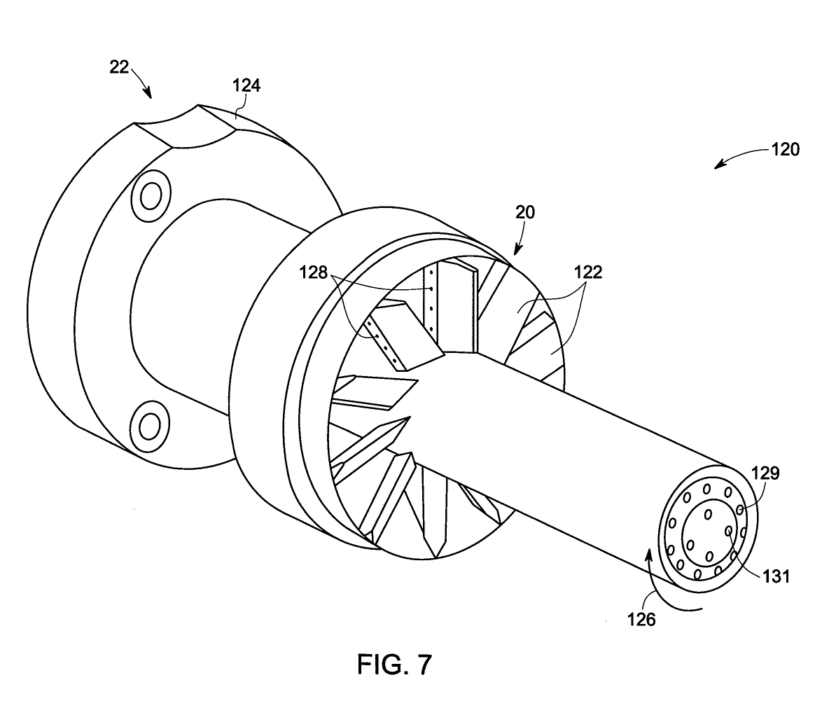

FIG. 7 is a diagrammatical illustration of an exemplary configuration 120 of the premixing device 100 (see FIG. 6) having a flow conditioning device 122. In the embodiment illustrated in FIG. 7, the flow conditioning device 122 includes a plurality of swirler vanes to provide a swirling motion to the fuel stream 22. In this embodiment, the fuel stream 22 includes natural gas. The fuel stream 22 enters the premixing device 120 through an inlet 124 located upstream of the swirler vanes 122. In this embodiment, the number of swirler vanes 122 is between about 4 to about 15. Furthermore, a turning angle of the swirler vanes 122 is between about 20 degrees to about 50 degrees. The swirler vanes 122 are configured to provide a swirl movement to the fuel stream 22 in a direction of rotation 126. Subsequently, oxygen 20 is introduced through a plurality of holes 128 located on the swirler vanes 122. In the illustrated embodiment, oxygen 20 is introduced in a transverse direction to the direction of fuel stream 22. Alternatively, oxygen 20 may be introduced at an angle to the direction of the fuel stream 22. It should be noted that the total effective hole area for introducing oxygen 20 is about 1/2 of the effective area of the swirler vanes 122. In one embodiment, the diameter of each of the plurality of holes 128 is between about 0.01 inches to about 0.04 inches. Further, a pitch of the holes 128 is between about 2 to about 10 times the diameter of the holes 128. In one embodiment, the pitch is about 5 times the diameter of the holes 128.

The fuel stream 22 and oxygen 20 are premixed to form the gaseous pre-mix that is directed to the combustion chamber 112 (see FIG. 6) through an exit that may be a straight or converging exit. The gaseous pre-mix is combusted at elevated temperature and pressure in the combustion chamber 112 to form syngas that is directed to a downstream process for further processing. In certain embodiments, a portion of oxygen 20 may be injected directly into the combustion chamber 112 to enhance the flashback resistance of the syngas generator. In certain other embodiments, fuel mixture may be injected through an outer ring of holes 129 on the tip. Further, oxygen 20 may also be injected through the tip through an inner set of holes 131. Such holes 129, 131 on the tip may be fed by internal passages in the tip. As described before, in certain embodiments, the fuel stream 22 may be augmented by steam 60 or a tail gas 58 from a downstream process. In certain other embodiments, steam 60 is premixed with oxygen 20 upstream of the premixing region 110 (see FIG. 6) thereby improving the resistance to autoignition and flashback in the premixing region 110.

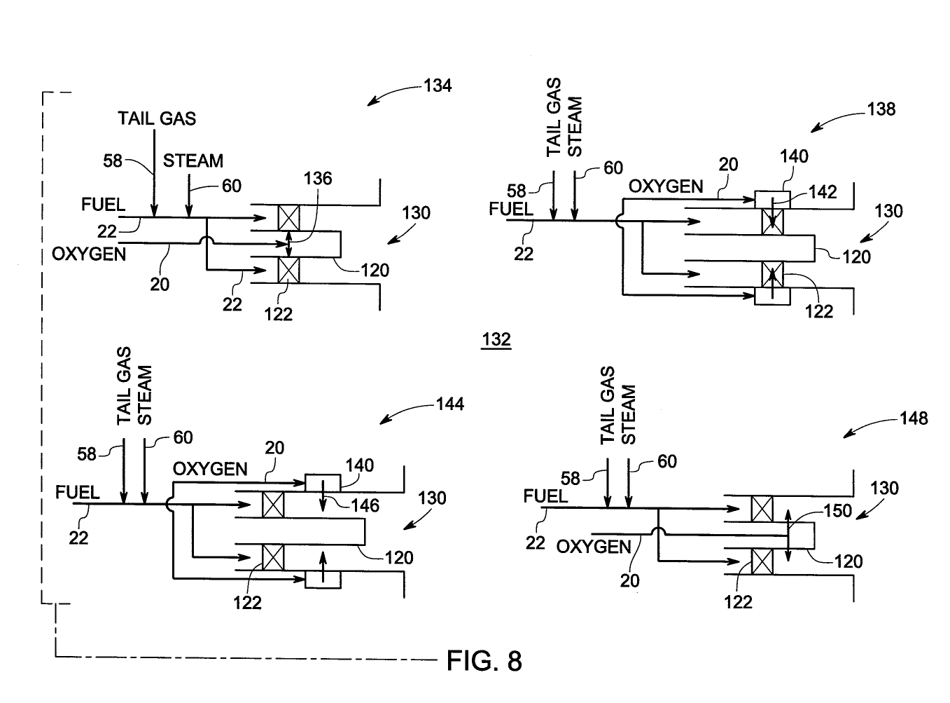

FIG. 8 is a diagrammatical illustration of exemplary configurations 132 of the premixing device 120 of FIG. 7 having different oxygen injection locations. For example, in an exemplary configuration 134, the fuel stream 22 introduced in the premixing device 134 is pre-conditioned via the swirler vanes 122. Further, oxygen 20 is introduced within the premixing device 134 through the swirler vanes 122 in a transverse direction to the direction of fuel stream 22, as represented by reference numeral 136. Again, as described before, the fuel stream 22 may be augmented by tail gas 58 or steam 60. The fuel stream 22 and oxygen 20 are premixed to form the gaseous pre-mix that is directed to the combustion chamber 112 (see FIG. 6) through an exit 130.

In another exemplary configuration 138, the fuel stream 22 is similarly introduced and pre-conditioned via the swirler vanes 122. Further, oxygen 20 is injected through holes 140 disposed on the burner tube, as represented by reference numeral 142. In particular, the oxygen 20 is injected through the burner tube into the swirler vanes 122 in a transverse direction to the direction of the fuel stream 22. In an alternate embodiment represented by reference numeral 144, the oxygen 20 is injected through the burner tube at injection points 140 disposed downstream of the swirler vanes 122. Thus, the oxygen 20 is injected in a transverse direction 146 to the direction of the fuel stream 22 via the injection points 140. Further, as illustrated in configuration 148, the oxygen 20 is introduced through the center body of the premixing device 148 and is injected in a transverse direction at a location downstream of the swirler vanes 122, as represented by reference numeral 150.

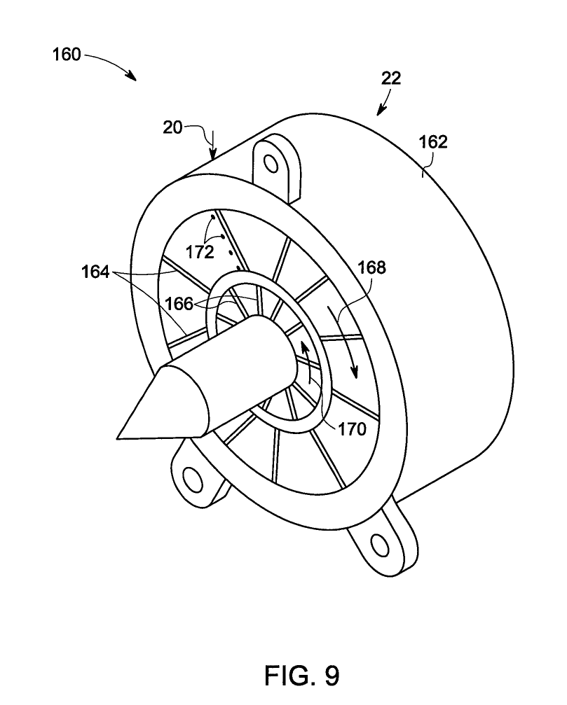

FIG. 9 is a diagrammatical illustration of another exemplary configuration 160 of the premixing device 100 of FIG. 6. As illustrated, the premixing device 160 includes a fuel inlet 162 to introduce the fuel stream 22 within the premixing device 160. Further, the premixing device 160 includes a plurality of swirler vanes 164 to provide a swirl movement to the fuel stream 22. Additionally, the premixing device 160 includes a plurality of counter flow swirl vanes 166 disposed adjacent to the plurality of swirler vanes 164. The direction of movement of the swirl and counter flow swirl vanes 164 and 166 is represented by reference numerals 168 and 170 respectively. In this exemplary embodiment, the fuel stream 22 flows from the inlet 162 upstream of the swirler vanes 164. Further, oxygen 20 is introduced through a plurality of holes 172 disposed on the swirler vanes 164.

In this embodiment, total effective area for the plurality of holes 172 is about 1/2 of the effective area of the swirler vanes 164. Further, the number of swirler vanes 164 is between about 4 to about 15. Similarly, the number of counter flow swirler vanes 166 is between about 4 to about 15. Additionally, the turning angle for each of the swirler vanes 164 and 166 is between about 20 degrees to about 55 degrees. In one embodiment, the turning angle of the counter flow swirler vanes 166 is relatively greater than the turning angle of the swirler vanes 164. As described earlier, the fuel stream 22 is pre-conditioned through the swirler vanes 164 and 166 and oxygen 20 is premixed with the pre-conditioned fuel stream to form a gaseous pre-mix that is directed to the combustion chamber 112 (see FIG. 6) through an exit 174. Subsequently, the gaseous pre-mix is combusted at elevated temperature and pressure in the combustion chamber 112 to form syngas.

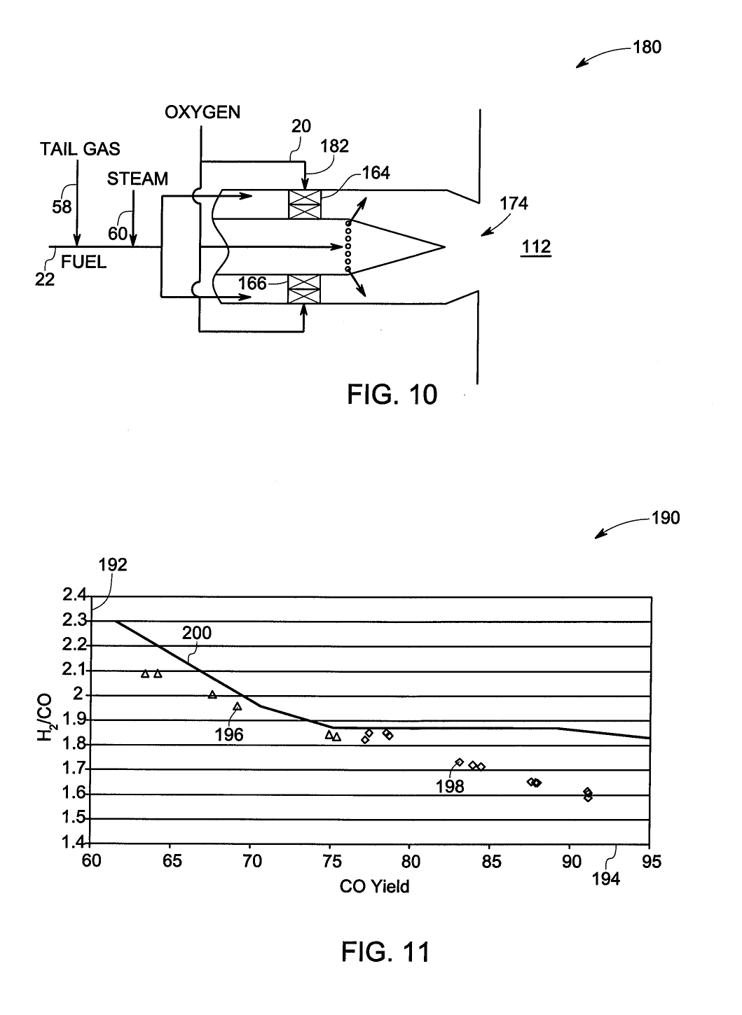

FIG. 10 is a diagrammatical illustration of an exemplary configuration 180 of the premixing device of FIG. 9. In this exemplary embodiment, the fuel stream 22 is introduced and is pre-conditioned via the swirler vanes 164. Further, the premixing device 180 also includes counter flow swirl vanes 166 disposed adjacent to the plurality of swirler vanes 164. As illustrated, oxygen 20 is introduced 182 through the swirler vanes 164 and is mixed with the pre-conditioned fuel stream 22 to form the gaseous pre-mix which is subsequently combusted in the combustion chamber 112 to form syngas. It should be noted that the mixing region could be either straight or converging prior to the exit 174. Further, oxygen 20 can also be introduced through the centerbody with an aerodynamic tip to prevent flow separation.

FIG. 11 represents exemplary results 190 illustrating H2:CO ratio 192 with % carbon monoxide (CO) yield 194 for the syngas generator 12 of FIG. 1. The percent of fuel carbon provided to the system that is converted to CO is the % CO yield. In this exemplary embodiment the syngas generator 12 is operated at a pressure of about 26 atmospheres and does not include any preheating or steam augmentation. The H2:CO ratio at two different residence times less than 100 ms is represented by reference numerals 196 and 198 and the equilibrium level, i.e. very long residence times, is represented by reference numeral 200. As can be seen, a high % CO yield can be obtained after a short residence time.

The various aspects of the method described hereinabove have utility in different applications such as syngas generators employed in gas to liquid systems. As noted above, the syngas generator based upon premixed partial oxidation combustion operates with a substantially high fuel to oxidizer ratio resulting in a syngas composition enriched with carbon monoxide and hydrogen. Further, the flow conditioning of the fuel stream and subsequent introduction of oxygen enables fast mixing of the fuel stream and oxygen thereby resulting in substantially shorter premixing residence time. Advantageously, such premixing of the fuel stream and oxygen prevents explosion of premixed natural gas and oxygen by minimizing the residence time and volume of the premixing region. Moreover, the combustion of substantially premixed reactants in the syngas generator leads to a compact reaction zone that achieves near-equilibrium composition and negligible formation of solid carbon in the reaction zone.

The premixing of the reactants prior to combustion as described above along with staging and piloting to optimize the operability and product composition enables a compact POₓ syngas generator. Advantageously, the premixed rich partial oxidation combustion substantially reduces the capital cost by reducing the size and complexity of the syngas generator. As will be appreciated by one skilled in the art the POₓ syngas generator described above may be developed through modular components independent of the gas to liquid plant where the syngas generator may be employed. Moreover, the compact size of the syngas generator also makes it desirable for use in GTL plants having limited space.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A premixed partial oxidation (POx) syngas generator, comprising:
a premixing device configured to mix a fuel stream and oxygen in a premixing region to form a gaseous pre-mix; wherein the premixing device comprises:
a fuel inlet configured to introduce the fuel stream within the premixing device;
a flow conditioning device configured to pre-condition the fuel stream;
and
an oxygen inlet configured to introduce oxygen into the fuel stream to facilitate premixing of the fuel stream and oxygen in the premixing region located downstream of the flow conditioning device; and
a combustion chamber configured to combust the gaseous pre-mix from the premixing device to produce a synthesis gas enriched with carbon monoxide and hydrogen gas.

2. The syngas generator of claim 1, wherein the flow conditioning device comprises a plurality of swirler vanes to provide a swirl movement to the fuel stream.

3. The syngas generator of claim 2, wherein the number of swirler vanes is between about 4 to about 15 and a turning angle for each of the swirler vanes is between about 20 degrees to about 55 degrees.

4. The premixing device of claim 2, wherein the flow conditioning device comprises a plurality of counter flow swirler vanes disposed adjacent and radially inward to the plurality of the swirler vanes.

5. The premixing device of claim 4, wherein the turning angle of each of the counter swirler vanes is relatively greater than the turning angle of each of the swirler vanes.

6. The syngas generator of claim 2, wherein the oxygen inlet comprises a plurality of holes disposed on each of the swirler vanes, or a center body, or walls of the premixing device, or combinations thereof.

7. The syngas generator of claim 1, wherein the flow conditioning device comprises a nozzle configured to accelerate the fuel stream to a desired velocity.

8. The syngas generator of claim 1, wherein the syngas generator comprises a rich premixed natural gas combustion system.

9. The syngas generator of claim 8, wherein the flow conditioning device is configured to introduce the fuel stream in the rich premixed natural gas combustion system and wherein oxygen is introduced in about 1/2 portions by volume.

10. The syngas generator of claim 1, further comprising a first inlet configured to introduce steam, a second inlet configured to introduce carbon dioxide (CO2), a third inlet configured to introduce tail gas into the fuel stream, a fourth inlet configured to introduce oxygen through holes in the swirler vanes, or centerbody, or walls of the premixing device, or combinations thereof.

11. The syngas generator of claim 10, wherein the tail gas is recirculated into the fuel stream from a downstream process of a syngas generator.

12. The syngas generator of claim 1, wherein a temperature of the fuel stream is between about 400° F. to about 1300° F. and a temperature of the oxygen stream is between about 200° F. to about 500° F.

13. The syngas generator of claim 1, wherein a ratio of an effective area of the oxygen inlet and an effective area of the flow conditioning device is between about 0.1 to about 0.5.

14. The syngas generator of claim 1, wherein a premixing residence time of the syngas generator is between about 0.25 ms to about 100 ms.

15. The syngas generator of claim 1, wherein the oxygen inlet is configured to introduce oxygen from the centerbody with a velocity component transverse to the fuel flow.

16. The syngas generator of claim 1, wherein the oxygen inlet is configured to introduce oxygen from the outer wall with a velocity component transverse to the fuel flow.

17. The syngas generator of claim 1, wherein the oxygen inlet is configured to introduce oxygen through a plurality of holes in the swirler vanes with a velocity component transverse to the fuel flow, or a velocity component tangential to the swirler vanes, or combinations thereof.

18. The syngas generator of claim 1, wherein the oxygen inlet is configured to introduce oxygen through the centerbody, or the outer wall, or the swirler vanes, or combinations thereof.

19. The syngas generator of claim 1, wherein the fuel stream is introduced through an outer ring of holes disposed on the tip of the premixing device and oxygen is introduced through an inner ring of holes disposed on the tip of the premixing device.

20. The syngas generator of claim 1, wherein a ratio of number of oxygen atoms to number of carbon atoms in the fuel stream in the premixing region is between about 0.6 to about 1.6.

21. The syngas generator of claim 1, wherein the syngas generator comprises a plurality of oxygen inlets to facilitate staging of oxygen flow within the syngas generator to enable substantially stable combustion.

22. The syngas generator of claim 1, wherein the combustion chamber is treated with a catalytic surface to promote syngas formation.

23. A gas to liquid system, comprising:
an air separation unit configured to separate oxygen from air;
a gas processing unit configured to prepare a fuel stream for combustion;
a combustion chamber for reacting oxygen with the fuel stream at an elevated temperature and pressure to produce a synthesis gas enriched with carbon monoxide and hydrogen gas; and
a premixing device disposed upstream of the combustion chamber and configured to mix the fuel stream and oxygen, wherein the premixing device comprises:
a fuel inlet configured to introduce the fuel stream within the premixing device;
a flow conditioning device configured to pre-condition the fuel stream; and
an oxygen inlet configured to introduce oxygen into the fuel stream to facilitate premixing of fuel stream and oxygen in a premixing region located downstream of the flow conditioning device.

24. The gas to liquid system of claim 23, further comprising a Fischer-Tropsch processing unit for receiving quenched synthesis gas and for catalytically converting the quenched synthesis gas into a long-chain hydrocarbon fluid.

25. The gas to liquid system of claim 24, further comprising an upgrading unit for fractionating the long-chain hydrocarbon fluid into at least one useful product.

26. The gas to liquid system of claim 25, wherein the at least one useful product comprises synthetic diesel fuel, or synthetic kerosene, or ethanol, or dimethyl ether, or naptha, or combinations thereof.

27. The gas to liquid system of claim 23, wherein the fuel stream comprises natural gas, or natural gas and tail gas, or natural gas and steam, or natural gas and tail gas and steam or natural gas and tail gas and CO2 or natural gas and tail gas and steam and CO2 or natural gas and steam and CO2.

28. The gas to liquid system of claim 23, wherein the flow conditioning device comprises a plurality of swirler vanes to provide a swirl movement to the fuel stream, or a nozzle configured to accelerate the fuel stream to a desired velocity.

28. The gas to liquid system of claim 27, wherein the oxygen inlet comprises a plurality of holes disposed on the swirler vanes, or a center body, or walls of the premixing device.

30. The gas to liquids system of claim 23, wherein the combustion chamber is treated with a catalytic surface to promote syngas formation in the reaction zone.

31. The gas to liquid system of claim 23, wherein the premixing device further comprises a first inlet configured to introduce steam into the fuel stream and a second inlet configured to introduce a tail gas into the fuel stream, and a third inlet configured to introduce CO2 into the fuel stream, and a fourth inlet configured to introduce an O2 stream downstream of the fuel flow conditioning device to facilitate premixing.

32. A method of generating a synthesis gas, comprising
introducing a fuel stream within a premixing device;
preconditioning the fuel stream through a flow conditioning device;
introducing an oxygen stream downstream of the flow conditioning device to facilitate premixing of the fuel stream and oxygen to form a gaseous pre-mix; and
forming the synthesis gas in a combustion chamber through partial oxidation of the gaseous pre-mix.

33. The method of claim 32, further comprising catalytically converting the quenched synthesis gas into a long-chain hydrocarbon fluid through a Fischer-Tropsch processing unit.

34. The method of claim 33, further comprising fractionating the long-chain hydrocarbon fluid into at least one useful product.

35. The method of claim 34, further comprising introducing steam, or a tail gas in the fuel stream within the premixing device.

36. The method of claim 33, further comprising recirculating a tail gas from the Fischer-Tropsch processing unit into the premixing device.

37. The method of claim 32, wherein preconditioning the fuel stream comprises generating a swirl movement in the fuel stream through a plurality of swirler vanes, or accelerating the fuel stream to a desired velocity through a nozzle.

38. The method of claim 32, comprising introducing oxygen with a velocity component transverse to the fuel stream, or a velocity component tangential to the center body, or combinations thereof.

39. The method of claim 32, further comprising introducing steam into the fuel stream within the premixing device to enhance the flashback resistance.
